(19) United States
(12) Patent Application Publication
(10) Pub. No.: US 2019/0231735 A1
(43) Pub. Date: Aug. 1, 2019

(54) METHODS FOR TREATING CANCER

(71) Applicant: Boston Biomedical, Inc., Cambridge, MA (US)

(72) Inventors: Chiang Jia Li, Cambridge, MA (US); Wei Li, Wayland, MA (US); Youzhi Li, Westwood, MA (US); Laura Borodyansky, Brookline, MA (US); Yuan Gao, Belmont, MA (US); David P. Kerstein, North Reading, MA (US)

(21) Appl. No.: 16/363,427

(22) Filed: Mar. 25, 2019

Related U.S. Application Data
(63) Continuation of application No. 15/567,061, filed on Oct. 16, 2017, now abandoned.
(60) Provisional application No. 62/281,022, filed on Jan. 20, 2016.
(60) Provisional application No. 62/149,349, filed on Apr. 17, 2015.

(51) Int. Cl. A61K 31/343; A61K 39/395; A61P 35/00; A61K 31/437; A61K 31/513; A61K 31/522

(52) U.S. Cl. CPC A61K 31/343; A61K 39/3955; A61K 31/522; A61K 31/437; A61K 31/513; A61P 35/00

(57) ABSTRACT

Methods comprising administration of, and kits comprising, at least one compound of formula (I)

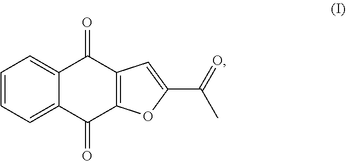 [SMILES: CC(=O)c1cc2c(o1)C(=O)c1ccccc1C2=O]

FOLFIRI, and optionally at least one angiogenesis inhibitor.

This application is a continuation of U.S. patent application Ser. No. 15/567,061, filed on Oct. 16, 2017, which is a U.S. national stage application filed under 35 U.S.C. § 371 from International Application No. PCT/US2016/028178, filed Apr. 18, 2016, which claims the benefit of priority under 35 U.S.C. § 119 of U.S. Provisional Patent Application No. 62/149,349, filed Apr. 17, 2015, and U.S. Provisional Patent Application No. 62/281,022, filed Jan. 20, 2016; the content of each respective application is incorporated herein by reference.

Disclosed herein are methods comprising administering to a subject a combination comprising a therapeutically effective amount of at least one compound of formula (I) in combination with a therapeutically effective amount of at least one 5-fluorouracil compound chosen from 5-fluorouracil, pharmaceutically acceptable salts thereof, and solvates of any of the foregoing; at least one irinotecan compound chosen from irinotecan, pharmaceutically acceptable salts thereof, and solvates of any of the foregoing; and at least one leucovorin compound, pharmaceutically acceptable salts thereof, and solvates of any of the foregoing (the combination of which components will be referred to as “FOLFIRI,” as defined below), and optionally at least one angiogenesis inhibitor.

The at least one compound of formula (I) is chosen from compounds having formula (I)

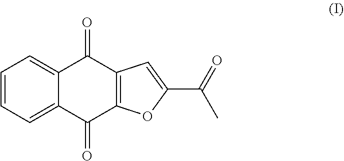 [SMILES: CC(=O)c1cc2c(o1)C(=O)c1ccccc1C2=O]

prodrugs, derivatives, pharmaceutically acceptable salts of any of the foregoing, and solvates of any of the foregoing.

Cancer fatalities in the United States alone number in the hundreds of thousands each year. Despite advances in the treatment of certain forms of cancer through surgery, radiotherapy, and chemotherapy, many types of cancer are essentially incurable. Even when an effective treatment is available for a particular cancer, the side effects of such treatment can be severe and result in a significant decrease in quality of life.

Most conventional chemotherapy agents have toxicity and limited efficacy, particularly for patients with advanced solid tumors. Conventional chemotherapeutic agents cause damage to non-cancerous as well as cancerous cells. The therapeutic index (i.e., a measure of a therapy's ability to discriminate between cancerous and normal cells) of such chemotherapeutic compounds can be quite low. Frequently, a dose of a chemotherapy drug that is effective to kill cancer cells will also kill normal cells, especially those normal cells (such as epithelial cells and cells of the bone marrow) that undergo frequent cell division. When normal cells are affected by the therapy, side effects such as hair loss, suppression of hematopoiesis, and nausea can occur. Depending on the general health of a patient, such side effects can preclude the administration of chemotherapy, or, at least, be extremely unpleasant and uncomfortable for the patient and severely decrease quality of the remaining life of cancer patients. Even for cancer patients who respond to chemotherapy with tumor regression, cancers often quickly relapse, progress and form more metastasis after initial response to chemotherapy. Such recurrent cancers become highly resistant or refractory to chemotherapeutics. As discussed below, cancer stem cells (CSCs) or cancer cells with high stemness (stemness-high cancer cells) are responsible for the rapid tumor recurrence and resistance to further traditional chemotherapy.

CSCs are believed to possess the following four characteristics:

1. Stemness—As used herein, stemness means the capacity to self-renew and differentiate into cancer cells (Gupta P B et al., Nat. Med. 2009; 15(9):1010-1012). While CSCs are only a minor portion of the total cancer cell population (Clarke M F, Biol. Blood Marrow Transplant. 2009; 11(2 suppl 2):14-16), they can give rise to heterogeneous lineages of cancer cells that make up the bulk of the tumor (see Gupta et al. 2009). In addition, CSCs possess the ability to mobilize to distinct sites while retaining their stemness properties and thus regrowth of the tumor at these sites (Jordan C T et al. N. Engl. J. Med. 2006; 355(12):1253-1261).

2. Aberrant signaling pathways—CSC stemness is associated with dysregulation of signaling pathways, which may contribute to their ability to regrow tumors and to migrate to distant sites. In normal stem cells, stemness signaling pathways are tightly controlled and genetically intact. In contrast, stemness signaling pathways in CSCs are dysregulated, allowing these cells to self-renew and differentiate into cancer cells (see Ajani et al. 2015). Dysregulation of stemness signaling pathways contributes to CSC resistance to chemotherapy and radiotherapy and to cancer recurrence and metastasis. Exemplary stemness signaling pathways involved in the induction and maintenance of stemness in CSCs include: JAK/STAT, Wnt/β-catenin, Hedgehog, Notch, and Nanog (Boman B M et al., J. Clin. Oncol. 2008; 26(17):2828-2838).

3. Resistance to traditional therapies—evidence suggests that CSCs possess resistance to conventional chemotherapy and radiation. While the detailed mechanism underlying such resistance is not well understood, the stemness pathways of CSCs (see Boman et al. 2008) together with the tumor microenvironment and aberrant regulation of signaling pathways (Borovski T. et al., Cancer Res. 2011; 71(3):634-639) may contribute to such resistance.

4. Ability to contribute to tumor recurrence and metastasis—although chemotherapy and radiation may kill most of the cells in a tumor, since CSCs are resistant to traditional therapies, the CSCs that are not eradicated may lead to regrowth or recurrence of the tumor either at the primary site or at distant sites (see Jordan et al. 2006). As mentioned above, CSCs may acquire the ability to mobilize to different sites and may maintain stemness at these sites through interactions with the microenvironment, allowing for metastatic tumor growth (see Boman et al. 2008).

The transcription factor Signal Transducer and Activator of Transcription 3 (referred to herein as Stat3) is a member of the Stat family, which are latent transcription factors activated in response to cytokines/growth factors to promote proliferation, survival, and other biological processes. Stat3 is an oncogene that can be activated by phosphorylation of a critical tyrosine residue mediated by growth factor receptor tyrosine kinases, including but not limited to, e.g., Janus kinases (JAKs), Src family kinases, EGFR, Abl, KDR, c-Met, and Her2. Yu, H. Stat3: Linking oncogenesis with tumor immune evasion in AACR 2008 Annual Meeting. 2008. San Diego, Calif. Upon tyrosine phosphorylation, the phosphorylated Stat3 (“pStat3”) forms homo-dimers and translocates to the nucleus, where it binds to specific DNA-response elements in the promoters of target genes, and induces gene expression. Pedranzini, L., et al. J. Clin. Invest., 2004. 114(5): p. 619-22.

In normal cells, Stat3 activation is transient and tightly regulated, lasting for example from 30 minutes to several hours. However, Stat3 is found to be aberrantly active in a wide variety of human cancers, including all the major carcinomas as well as some hematologic tumors. Persistently active Stat3 occurs in more than half of breast and lung cancers, colorectal cancers (CRC), ovarian cancers, hepatocellular carcinomas, multiple myelomas, etc., and in more than 95% of head/neck cancers. Stat3 plays multiple roles in cancer progression and is considered to be one of the major mechanisms for drug resistance to cancer cells. As a potent transcription regulator, Stat3 targets genes involved in cell cycle, cell survival, oncogenesis, tumor invasion, and metastasis, such as Bcl-xl, c-Myc, cyclin D1, Vegf, MMP-2, and survivin. Catlett-Falcone, R., et al. Immunity, 1999. 10(1): p. 105-15; Bromberg, J. F., et al. Cell, 1999. 98(3): p. 295-303; Kanda, N., et al. Oncogene, 2004. 23(28): p. 4921-29; Schlette, E. J., et al. J Clin Oncol, 2004. 22(9): p. 1682-88; Niu, G., et al. Oncogene, 2002. 21(13): p. 2000-08; Xie, T. X., et al. Oncogene, 2004. 23(20): p. 3550-60. It is also a key negative regulator of tumor immune surveillance and immune cell recruitment. Kortylewski, M., et al. Nat. Med., 2005. 11(12): p. 1314-21; Burdelya, L., et al. J. Immunol., 2005. 174(7): p. 3925-31; and Wang, T., et al. Nat. Med., 2004. 10(1): p. 48-54.

Abrogation of Stat3 signaling by using anti-sense oligonucleotides, siRNA, dominant-negative form of Stat3, and/or the targeted inhibition of tyrosine kinase activity causes cancer cell-growth arrest, apoptosis, and reduction of metastasis frequency both in vitro and/or in vivo. Pedranzini, L., et al. J Clin. Invest., 2004. 114(5): p. 619-22; Bromberg, J. F., et al. Cell, 1999. 98(3): p. 295-303; Darnell, J. E. Nat. Med., 2005. 11(6): p. 595-96; and Zhang, L., et al. Cancer Res, 2007. 67(12): p. 5859-64.

Furthermore, Stat 3 may play a role in the survival and self-renewal capacity of CSCs across a broad spectrum of cancers. Therefore, an agent with activity against CSCs may hold great promise for cancer patients (Boman, B. M., et al. J. Clin. Oncol. 2008. 26(17): p. 2795-99).

As discussed above, CSCs are a sub-population of cancer cells (found within solid tumors or hematological cancers) that possess characteristics normally associated with stem cells. These cells can grow faster after reduction of non-stem regular cancer cells by chemotherapy, which may be the mechanism for quick relapse after chemotherapies. In contrast to the bulk of cancer cells, which are non-tumorigenic, CSCs are tumorigenic (tumor-forming). In human acute myeloid leukemia, the frequency of these cells is less than 1 in 10,000. Bonnet, D. and J. E. Dick. Nat. Med., 1997. 3(7): p. 730-37. There is mounting evidence that such cells exist in almost all tumor types. However, as cancer cell lines are selected from a sub-population of cancer cells that are specifically adapted to growth in tissue culture, the biological and functional properties of these cell lines can change dramatically. Therefore, not all cancer cell lines contain CSCs.

CSCs have stem cell properties such as self-renewal and the ability to differentiate into multiple cell types. They persist in tumors as a distinct population and they give rise to the differentiated cells that form the bulk of the tumor mass and phenotypically characterize the disease. CSCs have been demonstrated to be fundamentally responsible for carcinogenesis, cancer metastasis, cancer recurrence, and relapse. CSCs are also called, for example, tumor initiating cells, cancer stem-like cells, stem-like cancer cells, highly tumorigenic cells, or super malignant cells.

CSCs are inherently resistant to conventional chemotherapies, which means they are left behind by conventional therapies that kill the bulk of tumor cells. As such, the existence of CSCs has several implications in terms of cancer treatment and therapy. These include, for example, disease identification, selective drug targets, prevention of cancer metastasis and recurrence, treatment of cancer refractory to chemotherapy and/or radiotherapy, treatment of cancers inherently resistant to chemotherapy or radiotherapy and development of new strategies in fighting cancer.

The efficacy of cancer treatments are, in the initial stages of testing, often measured by the amount of tumor mass they kill off. As CSCs form a very small proportion of the tumor cell population and have markedly different biologic characteristics than their differentiated progeny, the measurement of tumor mass may not select for drugs that act specifically on the stem cells. In fact, CSCs are radio-resistant and refractory to chemotherapeutic and targeted drugs. Normal somatic stem cells are naturally resistant to chemotherapeutic agents—they have various pumps (e.g., multidrug resistance protein pump) that efflux drugs, higher DNA repair capability, and have a slow rate of cell turnover (chemotherapeutic agents naturally target rapidly replicating cells). CSCs, being the mutated counterparts of normal stem cells, may also have similar functions that allow them to survive therapy. In other words, conventional chemotherapies kill differentiated (or differentiating) cells, which form the bulk of the tumor that is unable to generate new cells. A population of CSCs that gave rise to the tumor could remain untouched and cause a relapse of the disease. Furthermore, treatment with chemotherapeutic agents may only leave chemotherapy-resistant CSCs, so that the ensuing tumor will most likely also be resistant to chemotherapy. Cancer stem cells have also been demonstrated to be resistant to radiation therapy (XRT). Hambardzumyan, et al. Cancer Cell, 2006. 10(6): p. 454-56; and Baumann, M., et al. Nat. Rev. Cancer, 2008. 8(7): p. 545-54.

Since surviving CSCs can repopulate the tumor and cause relapse, anti-cancer therapies that include strategies against CSCs hold great promise. Jones R J et al., J Natl Cancer Inst. 2004; 96(8):583-585. By targeting CSC pathways, it may be possible to treat patients with aggressive, non-resectable tumors and refractory or recurrent cancers as well as prevent tumor metastasis and recurrence. Development of specific therapies targeting CSC pathways, therefore, may improve the survival and quality of life of cancer patients, especially those patients suffering from metastatic disease. Unlocking this untapped potential may involve the identification and validation of pathways that are selectively important for CSC self-renewal and survival. Though multiple pathways underlying tumorigenesis in cancer and in embryonic stem cells or adult stem cells have been elucidated in the past, pathways for cancer stem cell self-renewal and survival are still sought.

Methods for identification and isolation of CSCs have been reported. The methods used mainly exploit the ability of CSCs to efflux drugs or have been based on the expression of surface markers associated with cancer stem cells.

For example, since CSCs are resistant to many chemotherapeutic agents, it is not surprising that CSCs almost ubiquitously overexpress drug efflux pumps such as ABCG2 (BCRP-1), and other ATP binding cassette (ABC) superfamily members. Ho, M. M., et al. Cancer Res., 2007. 67(10): p. 4827-33; Wang, J., et al. Cancer Res., 2007. 67(8): p. 3716-24; Haraguchi, N., et al. Stem Cells, 2006. 24(3): p. 506-13; Doyle, L. A. and D. D. Ross. Oncogene, 2003. 22(47): p. 7340-58; Alvi, A. J., et al. Breast Cancer Res., 2003. 5(1): p. R1-R8; Frank, N. Y., et al. Cancer Res., 2005. 65(10): p. 4320-33; and Schatton, T., et al. Nature, 2008. 451(7176): p. 345-49. Accordingly, the side population (SP) technique, originally used to enrich hematopoetic and leukemic stem cells, was also employed to identify and isolate CSCs. Kondo, T., et al. Proc. Natl Acad. Sci. USA, 2004. 101(3): p. 781-86. This technique, first described by Goodell et al., takes advantage of differential ABC transporter-dependent efflux of fluorescent dyes such as Hoechst 33342 to define a cell population enriched in CSCs. Doyle, L. A. and D. D. Ross. Oncogene, 2003. 22(47): p. 7340-58; and Goodell, M. A., et al. J. Exp. Med., 1996. 183(4): p. 1797-806. Specifically, the SP is revealed by blocking drug efflux with verapamil, at which point the dyes can no longer be pumped out of the SP.

Efforts have also focused on finding specific markers that distinguish CSCs from the bulk of the tumor. Markers originally associated with normal adult stem cells have been found to also mark CSCs and co-segregate with the enhanced tumorigenicity of CSCs. Commonly expressed surface markers by the CSCs include CD44, CD133, and CD166. Al-Hajj, M., et al. Proc. Natl Acad. Sci. USA, 2003. 100(7): p. 3983-88; Collins, A. T., et al. Cancer Res., 2005. 65(23): p. 10946-51; Li, C., et al. Cancer Res., 2007. 67(3): p. 1030-37; Ma, S., et al. Gastroenterology, 2007. 132(7): p. 2542-56; Ricci-Vitiani, L., et al. Nature, 2007. 445(7123): p. 111-15; Singh, S. K., et al. Cancer Res., 2003. 63(18): p. 5821-28; and Bleau, A. M., et al., Neurosurg. Focus, 2008. 24(3-4): p. E28. Sorting tumor cells based primarily upon the differential expression of these surface marker(s) have accounted for the majority of the highly tumorigenic CSCs described to date. Therefore, these surface markers are validated for identification and isolation of CSCs from the cancer cell lines and from the bulk of tumor tissues.

By using aiRNA (asymmetric RNA duplexes), potent Stat3 selective silencing has been achieved in stemness-high cancer cells. This Stat3 silencing may lead to downregulation of cancer cell stemness, and/or inhibition of stemness-high cancer cell survival and self-renewal.

In some embodiments, the at least one compound of formula (I) is an inhibitor of CSC growth and survival. According to U.S. Pat. No. 8,877,803, the compound of formula (I) inhibits Stat3 pathway activity with a cellular IC₅₀ of ˜0.25 μM. The at least one compound of formula (I) may be synthesized according to U.S. Pat. No. 8,877,803, for example, Example 13. In some embodiments, the at least one compound of formula (I) is used in a method of treating cancers. According to PCT Patent Application No. PCT/US2014/033566, Example 6, the at least one compound of formula (I) was chosen to enter a clinical trial for patients with advanced cancers. The disclosures of U.S. Pat. No. 8,877,803 and PCT Patent Application No. PCT/US2014/033566 are incorporated herein by reference in their entireties.

We have surprisingly discovered that patients with higher expression levels of Stat3 show prolonged overall survival after treatment with at least one compound of formula (I) in clinical trials. Thus, the higher the level of pStat3 found in a cancer patient before treatment, at least in CRC patients, the higher the overall survival (OS) upon administering a treatment comprising a compound of formula (I).

We also have surprisingly discovered that a treatment combination of at least one compound of formula (I) with FOLFIRI, with or without at least one angiogenesis inhibitor, results in anti-tumor activity in patients with certain types of cancer that progressed on prior FOLFIRI treatment.

In some embodiments, disclosed herein are methods for treating cancer comprising administering to a subject in need thereof:

a therapeutically effective amount of at least one compound of formula (I) chosen from compounds having formula (I):

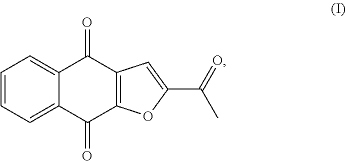 [SMILES: CC(=O)c1cc2c(o1)C(=O)c1ccccc1C2=O]

prodrugs, derivatives, pharmaceutically acceptable salts of any of the foregoing, and solvates of any of the foregoing, and
a therapeutically effective regimen of FOLFIRI, and
optionally at least one angiogenesis inhibitor, for example, bevacizumab, or a pharmaceutically acceptable salt or solvate thereof.

In some embodiments, disclosed herein are methods for treating cancer that had progressed on at least one prior FOLFIRI regimen comprising administering to a subject in need thereof:

a therapeutically effective amount of at least one compound of formula (I) chosen from compounds having formula (I)

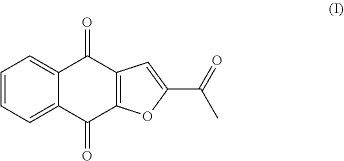 [SMILES: CC(=O)c1cc2c(o1)C(=O)c1ccccc1C2=O]

prodrugs, derivatives, pharmaceutically acceptable salts of any of the foregoing, and solvates of any of the foregoing,
a therapeutically effective regimen of FOLFIRI, and
optionally at least one angiogenesis inhibitor.

In some embodiments, disclosed herein are methods for simultaneously inhibiting, reducing, and/or diminishing (i) cancer stem cell survival and/or self-renewal, and (ii) survival and/or proliferation of heterogeneous cancer cells, comprising administering to a subject in need thereof:

a therapeutically effective amount of at least one compound of formula (I) chosen from compounds having formula (I)

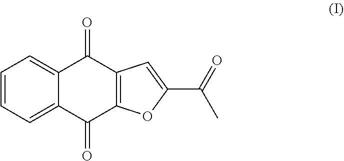 [SMILES: CC(=O)c1cc2c(o1)C(=O)c1ccccc1C2=O]

prodrugs, derivatives, pharmaceutically acceptable salts of any of the foregoing, and solvates of any of the foregoing,
a therapeutically effective regimen of FOLFIRI, and
optionally at least one angiogenesis inhibitor.

The at least one compound of formula (I), one or more of the components of FOLFIRI, and/or the at least one angiogenesis inhibitor may be administered to a subject simultaneously or sequentially.

In some embodiments, disclosed herein are methods for re-sensitizing a subject to FOLFIRI comprising administering to a subject whose cancer progressed on at least one prior therapy regimen:

a therapeutically effective amount of at least one compound chosen from compounds having formula (I)

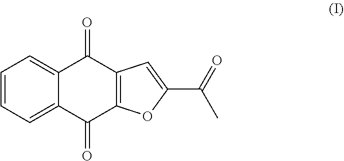 [SMILES: CC(=O)c1cc2c(o1)C(=O)c1ccccc1C2=O]

prodrugs, derivatives, pharmaceutically acceptable salts of any of the foregoing, and solvates of any of the foregoing. In some embodiments, the at least one prior therapy regimen is chosen from chemotherapy regimens. In some embodiments, the at least one prior therapy regimen is chosen from FOLFIRI regimens. In some embodiments, disclosed herein are methods for re-sensitizing a subject to FOLFIRI comprising administering to a subject whose cancer progressed on at least one prior FOLFIRI regimen:

a therapeutically effective amount of at least one compound chosen from compounds having formula (I)

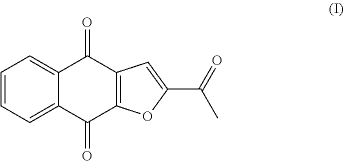 [SMILES: CC(=O)c1cc2c(o1)C(=O)c1ccccc1C2=O]

prodrugs, derivatives, pharmaceutically acceptable salts of any of the foregoing, and solvates of any of the foregoing.

In some embodiments, a kit is disclosed that comprises at least one compound of formula (I), at least one 5-fluorouracil compound chosen from 5-fluorouracil, pharmaceutically acceptable salts thereof, and solvates of any of the foregoing, at least one irinotecan compound chosen from irinotecan, pharmaceutically acceptable salts thereof, and solvates of any of the foregoing, and at least one leucovorin compound, chosen from leucovorin, pharmaceutically acceptable salts thereof, and solvates of any of the foregoing, and optionally at least one angiogenesis inhibitor, together with instructions for administration and/or use.

Aspects and embodiments of the present disclosure are set forth or will be readily apparent from the following detailed description. It is to be understood that both the foregoing general description and the following detailed description are exemplary and explanatory only, and are not intended to be restrictive of the claims.

### BRIEF DESCRIPTION OF THE FIGURES

FIG. 1 shows the Stat3 pathway in cancer.

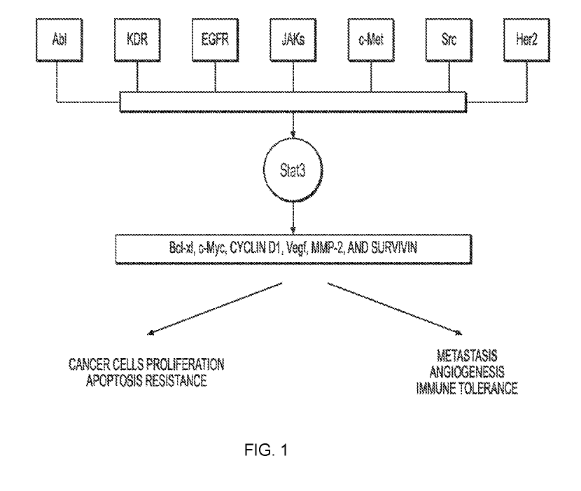

FIG. 2 shows the cancer stem cell specific and conventional cancer therapies.

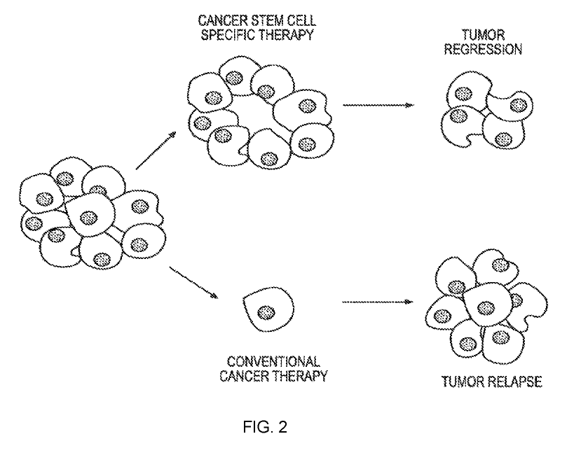

FIG. 3 shows the formation of heterogeneous cancer cells from cancer stem cells.

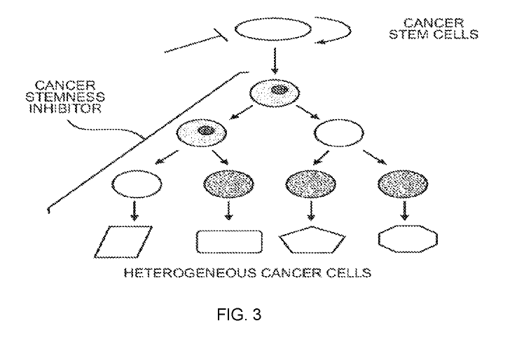

FIG. 4 shows the percent change in target lesions (best response) in colorectal cancer patients receiving FOLFIRI (8 patients) or FOLFIRI, bevacizumab, and 2-acetylnaphtho[2,3-b]furan-4,9-dione (9 patients). The x-axis shows the patient identification numbers.

FIG. 5A and FIG. 5B show exemplary CT scans of a patient before and after receiving an embodiment of the present disclosure.

The following are definitions of terms used in the present specification. The initial definition provided for a group or term herein applies to that group or term throughout the present specification individually or as part of another group, unless otherwise indicated.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

The terms “administer,” “administering,” or “administration” are used herein in their broadest sense. These terms refer to any method of introducing to a subject a compound or pharmaceutical composition described herein and can include, for example, introducing the compound systemically, locally, or in situ to the subject. Thus, a compound of the present disclosure produced in a subject from a composition (whether or not it includes the compound) is encompassed in these terms. When these terms are used in connection with the term “systemic” or “systemically,” they generally refer to in vivo systemic absorption or accumulation of the compound or composition in the blood stream followed by distribution throughout the entire body.

The term “subject” generally refers to an organism to which a compound or pharmaceutical composition described herein can be administered. A subject can be a mammal or mammalian cell, including a human or human cell. The term also refers to an organism, which includes a cell or a donor or recipient of such cell. In various embodiments, the term “subject” refers to any animal (e.g., a mammal), including, but not limited to humans, mammals and non-mammals, such as non-human primates, mice, rabbits, sheep, dogs, cats, horses, cows, chickens, amphibians, and reptiles, which is to be the recipient of a compound or pharmaceutical composition described herein. Under some circumstances, the terms “subject” and “patient” are used interchangeably herein in reference to a human subject.

The terms “effective amount” and “therapeutically effective amount” refer to that amount of a compound or pharmaceutical composition described herein that is sufficient to effect the intended result including, but not limited to, disease treatment, as illustrated below. In some embodiments, the “therapeutically effective amount” is the amount that is effective for detectable killing or inhibition of the growth or spread of cancer cells, the size or number of tumors, and/or other measure of the level, stage, progression and/or severity of the cancer. In some embodiments, the “therapeutically effective amount” refers to the amount that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose may vary depending on, for example, the particular pharmaceutical composition, subject and their age and existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “treatment,” “treating,” “ameliorating,” and “encouraging” may be used interchangeably herein. These terms refer to an approach for obtaining beneficial or desired results including, but not limited to, therapeutic benefit and/or prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical composition may be administered to a patient at risk of developing a particular disease, or to a patient reporting one or more of the physiological symptoms of a disease, even though a diagnosis of this disease may not have been made.

The term “cancer” in a subject refers to the presence of cells possessing characteristics typical of cancer-causing cells, such as uncontrolled proliferation, immortality, metastatic potential, rapid growth and proliferation rate, and certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, colon cancer, rectal cancer, cancer of the anal region, stomach cancer, gastric cancer, gastrointestinal cancer, gastric adenocarcinoma, adrenocorticoid carcinoma, uterine cancer, carcinoma of the fallopian tubes, carcinoma of the endometrium, carcinoma of the vagina, carcinoma of the vulva, Hodgkin's Disease, esophageal cancer, gastroesophageal junction cancer, gastroesophageal adenocarcinoma, chondrosarcoma, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and are included in this term. For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

Also included within the term “cancer” is “solid tumor.” As used herein, the term “solid tumor” refers to those conditions, such as cancer, that form an abnormal tumor mass, such as sarcomas, carcinomas, and lymphomas. Examples of solid tumors include, but are not limited to, non-small cell lung cancer (NSCLC), neuroendocrine tumors, thyomas, fibrous tumors, metastatic colorectal cancer (mCRC), and the like. In some embodiments, the solid tumor disease is an adenocarcinoma, squamous cell carcinoma, large cell carcinoma, and the like.

In some embodiments, the cancer is chosen from colon adenocacrinoma, rectal adenocarcinoma, gastric adenocarcinoma, gastroesophageal junction adenocarcinoma, esophageal adenocarcinoma, hepatocellular carcinoma, ovarian cancer, platinum-resistant ovarian cancer, pancreatic adenocarcinoma, breast cancer, triple negative breast cancer, ovarian cancer, cholangiocarcinoma, melanoma, small cell lung cancer, and non-small cell lung cancer. In some embodiments, the cancer is colorectal cancer. In some embodiments, the cancer is advanced colorectal cancer. In some embodiments, the cancer is gastric adenocarcinoma. In some embodiments, the cancer is colon adenocarcinoma. In some embodiments, the cancer is rectal adenocarcinoma.

The terms “progress,” “progressed,” and “progression” refer to at least one of the following: (1) a response to prior therapy (e.g., chemotherapy) of progressive disease (PD); (2) the appearance of one or more new lesions after treatment with prior therapy (e.g., chemotherapy); and (3) at least a 5% (e.g., 10%, 20%) increase in the sum of diameters of target lesions, taking as a reference the smallest sum on study (this includes the baseline sum if that is the smallest on study).

As used herein, “re-sensitizing” means making patients who were previously resistant, non-responsive, or somewhat responsive to a prior therapy (e.g., chemotherapy) regimen sensitive, responsive, or more responsive to that prior therapy (e.g., chemotherapy) regimen.

As used herein, the term “at least one compound of formula (I)” means a compound chosen from compounds having formula (I)

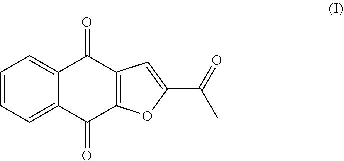 [SMILES: CC(=O)c1cc2c(o1)C(=O)c1ccccc1C2=O]

prodrugs, derivatives, pharmaceutically acceptable salts of any of the foregoing, and solvates of any of the foregoing. In some embodiments, prodrugs and derivatives of compounds having formula (I) are Stat3 inhibitors. Non-limiting examples of prodrugs of compounds having formula (I) are the phosphoric ester and phosphoric diester described in U.S. pre-grant Publication No. 2012/0252763 as compound numbers 4011 and 4012 and also suitable compounds described in U.S. Pat. No. 9,150,530. Non-limiting examples of derivatives of compounds having formula (I) include the derivatives disclosed in U.S. Pat. No. 8,877,803. The disclosures of U.S. pre-grant Publication No. 2012/0252763 and U.S. Pat. Nos. 9,150,530 and 8,877,803 are incorporated herein by reference in their entireties.

Compounds having formula (I), shown below,

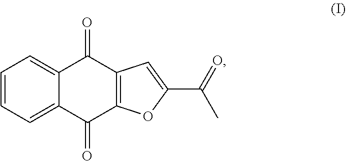 [SMILES: CC(=O)c1cc2c(o1)C(=O)c1ccccc1C2=O]

may also be known as 2-acetylnaphtho[2,3-b]furan-4,9-dione, napabucasin, or 661608 and include tautomers thereof.

Suitable methods of preparing 2-acetylnaphtho[2,3-b]furan-4,9-dione, including its crystalline forms and additional cancer stemness inhibitors, are described in the co-owned PCT applications published as WO 2009/036099, WO 2009/036101, WO 2011/116398, WO 2011/116399, and WO 2014/169078; the contents of each application is incorporated herein by reference.

The term “salt(s),” as used herein, includes acidic and/or basic salts formed with inorganic and/or organic acids and bases. As used herein, the term “pharmaceutically acceptable salt” refers to those salts which are, within the scope of sound medical judgment, suitable for use in contact with the tissues of subjects without undue toxicity, irritation, allergic response and/or the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

Pharmaceutically acceptable salts may be formed with inorganic or organic acids. Non-limiting examples of suitable inorganic acids include hydrochloric acid, hydrobromic acid, phosphoric acid, sulfuric acid, and perchloric acid. Non-limiting examples of suitable organic acids include acetic acid, oxalic acid, maleic acid, tartaric acid, citric acid, succinic acid, and malonic acid. Other non-limiting examples of suitable pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid.

Salts may be prepared in situ during the isolation and purification of the disclosed compound, or separately, such as by reacting the compound with a suitable base or acid, respectively. Non-limiting examples of pharmaceutically acceptable salts derived from bases include alkali metal, alkaline earth metal, ammonium and N+(C₁₋₄alkyl)₄ salts. Non-limiting examples of suitable alkali or alkaline earth metal salts include sodium, lithium, potassium, calcium, magnesium, iron, zinc, copper, manganese, and aluminum salts. Further non-limiting examples of suitable pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Non-limiting examples of suitable organic bases from which salts may be derived include primary amines, secondary amines, tertiary amines, substituted amines including naturally occurring substituted amines, cyclic amines, and basic ion exchange resins, such as isopropylamine, trimethylamine, diethylamine, triethylamine, tripropylamine, and ethanolamine. In some embodiments, pharmaceutically acceptable base addition salts can be chosen from ammonium, potassium, sodium, calcium, and magnesium salts.

The term “solvate” represents an aggregate that comprises one or more molecules of a compound of the present disclosure with one or more molecules of a solvent or solvents. Solvates of the compounds of the present disclosure include, for example, hydrates.

The term “FOLFIRI” as used herein refers to a combination therapy (e.g., chemotherapy) comprising at least one irinotecan compound chosen from irinotecan, pharmaceutically acceptable salts thereof, and solvates of any of the foregoing; at least one 5-fluorouracil (also known as 5-FU) compound chosen from 5-fluorouracil, pharmaceutically acceptable salts thereof, and solvates of any of the foregoing; and at least one compound chosen from folinic acid (also known as leucovorin), levofolinate (the levo isoform of folinic acid), pharmaceutically acceptable salts of any of the foregoing, and solvates of any of the foregoing. The term “FOLFIRI” as used herein is not intended to be limited to any particular amounts of or dosing regimens for these components. Rather, as used herein, “FOLFIRI” includes all combinations of these components in any amounts and dosing regimens. As used herein, any recitation of the term “FOLFIRI” may be replaced with a recitation of the individual components. For example, the term “FOLFIRI” may be replaced with the phrase “at least one irinotecan compound chosen from irinotecan, pharmaceutically acceptable salts of irinotecan, solvates of irinotecan, and solvates of pharmaceutically acceptable salts of irinotecan; at least one 5-fluorouracil compound chosen from 5-fluorouracil, pharmaceutically acceptable salts of 5-fluorouracil, solvates of 5-fluorouracil, and solvates of pharmaceutically acceptable salts of 5-fluorouracil; and at least one leucovorin compound chosen from leucovorin, pharmaceutically acceptable salts of leucovorin, solvates of leucovorin, and solvates of pharmaceutically acceptable salts of leucovorin.”

A “therapeutically effective regimen” of FOLFIRI, as used herein, means a therapeutically effective amount of the components of FOLFIRI as defined herein that is sufficient to effect the intended result including, but not limited to, disease treatment, as illustrated below. In various embodiments, a therapeutically effective regimen of FOLFIRI comprises a therapeutically effective amount of irinotecan, a pharmaceutically acceptable salt thereof, or a pharmaceutically acceptable solvate thereof; a therapeutically effective amount of folinic acid and/or levofolinate, a pharmaceutically acceptable salt thereof, or a pharmaceutically acceptable solvate thereof; and a therapeutically effective amount of fluorouracil (5-FU), a pharmaceutically acceptable salt thereof, or a pharmaceutically acceptable solvate thereof, where the term “a therapeutically effective amount” is as defined herein. In some embodiments, a therapeutically effective regimen of FOLFIRI comprises administration of a therapeutically effective amount of irinotecan simultaneously (separately or together) or successively with a therapeutically effective amount of folinic acid and/or levofolinate, followed by administration of a therapeutically effective amount of 5-FU. In some embodiments, the administration of at least one of the components (irinotecan, folinic acid and/or levofolinate, and 5-FU) includes a bolus injection. In some embodiments, the administration of at least one of the components (irinotecan, folinic acid and/or levofolinate, and 5-FU) includes infusion. In some embodiments, at least one of the components (irinotecan, folinic acid and/or levofolinate, and 5-FU) is administered in separate doses (e.g., two or more doses). For example, irinotecan can be administered in two or more separate doses with one dose before and another dose after the administration of another component (e.g., 5-FU). In the similar manner, for example, folinic acid and/or levofolinate can be administered in two or more separate doses with one dose before and another dose after the administration of another component (e.g., 5-FU). In some embodiments, a therapeutically effective regimen of FOLFIRI comprises intravenous administration of irinotecan (e.g., about 180 mg/m²) simultaneously (separately or together) or successively with levofolinate (e.g., about 200 mg/m²), followed by a bolus injection of 5-FU (e.g., about 400 mg/m²), and then by infusion of 5-FU (e.g., about 1200 mg/m²/day or total about 2400 mg/m²). In some embodiments, a therapeutically effective regimen of FOLFIRI comprises administration of irinotecan at 180 mg/m² IV over 90 minutes concurrently with folinic acid at 400 mg/m² (or 2×250 mg/m²) IV over 120 minutes, followed by 5-fluorouracil at 400-500 mg/m² in an IV bolus, then 5-fluorouracil at 2400-3000 mg/m² as an intravenous infusion over 46 hours. In some embodiments, a therapeutically effective regimen of FOLFIRI comprises intravenous administration of irinotecan (e.g., about 180 mg/m²) simultaneously (separately or together) or successively with levofolinate (e.g., about 200 mg/m²), followed by infusion of 5-FU (e.g., about 2400 mg/m²). In some embodiments, FOLFIRI is administered bi-weekly.

In some embodiments, the methods disclosed herein further comprise administering at least one angiogenesis inhibitor. In some embodiments, the at least one angiogenesis inhibitor is chosen from bevacizumab and pharmaceutically acceptable salts thereof. In some embodiments, the methods disclosed herein further comprise administering a therapeutically effective amount of at least one angiogenesis inhibitor. In some embodiments, bevacizumab (e.g., about 5 mg/kg) is administered intravenously following infusion of irinotecan and/or levofolinate/leucovorin. In some embodiments, bevacizumab is administered bi-weekly.

The at least one compound disclosed herein may be in the form of a pharmaceutical composition. In some embodiments, the pharmaceutical compositions may comprise the at least one compound of formula (I) and at least one pharmaceutically acceptable carrier. In some embodiments, the pharmaceutical compositions may comprise one or more compounds and at least one pharmaceutically acceptable carrier, where the one or more compounds are capable of being converted into the at least one compound chosen from compounds of formula (I) and pharmaceutically acceptable salts and solvates thereof in a subject (i.e., a prodrug).

The term “carrier” as used herein means a pharmaceutically acceptable material, composition or vehicle, such as, for example, a liquid or solid filler, diluent, excipient, solvent or encapsulating material involved in or capable of carrying or transporting the subject pharmaceutical compound from one organ, or portion of the body, to another organ, or portion of the body. Each carrier must be “acceptable” in the sense of being compatible with the other ingredients of the formulation and not injurious to the patient. Non-limiting examples of pharmaceutically acceptable carriers, carriers, and/or diluents include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

In some embodiments, the at least one compound may be administered in an amount ranging from about 160 to about 1500 mg. In some embodiments, the at least one compound may be administered in an amount ranging from about 160 to about 1000 mg. In some embodiments, the at least one compound may be administered in an amount ranging from about 300 mg to about 700 mg. In some embodiments, the at least one compound may be administered in an amount ranging from about 700 mg to about 1200 mg. In some embodiments, the at least one compound may be administered in an amount ranging from about 800 mg to about 1100 mg. In some embodiments, the at least one compound may be administered in an amount ranging from about 850 mg to about 1050 mg. In some embodiments, the at least one compound may be administered in an amount ranging from about 960 mg to about 1000 mg. In some embodiments, the total amount of the at least one compound is administered once daily. In some embodiments, the at least one compound is administered in a dose of about 480 mg daily. In some embodiments, the at least one compound is administered in administered in a dose of about 960 mg daily. In some embodiments, the at least one compound is administered in a dose of about 1000 mg daily. In some embodiments, the total amount of the at least one compound is administered in divided doses more than once daily, such as twice daily (BID) or more often. In some embodiments, the at least one compound may be administered in an amount ranging from about 80 to about 750 mg twice daily. In some embodiments, the at least one compound may be administered in an amount ranging from about 80 to about 500 mg twice daily. In some embodiments, the at least one compound is administered in a dose of about 240 mg twice daily. In some embodiments, the at least one compound is administered in a dose of about 480 mg twice daily. In some embodiments, the at least one compound is administered in a dose of about 500 mg twice daily.

Pharmaceutical compositions disclosed herein that are suitable for oral administration may be in the form of capsules, cachets, pills, tablets, lozenges (using a flavored basis, usually sucrose and acacia or tragacanth), powders, granules, a solution in an aqueous or non-aqueous liquid, a suspension in an aqueous or non-aqueous liquid, an oil-in-water emulsion, a water-in-oil emulsion, an elixir, a syrup, pastilles (using an inert base, such as gelatin, glycerin, sucrose, and/or acacia) and/or mouthwashes, each containing a predetermined amount of the at least one compound of the present disclosure.

A pharmaceutical composition disclosed herein may be administered as a bolus, electuary, or paste.

Solid dosage forms for oral administration (capsules, tablets, pills, dragees, powders, granules and the like) may be mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type also may be employed as fillers in soft and hard-filled gelatin capsules using such excipients as lactose or milk sugars, as well as high molecular weight polyethylene glycols and the like.

Liquid dosage forms for oral administration may include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxypropyl-6-cyclodextrin, may be used to solubilize compounds.

The pharmaceutical compositions also may include adjuvants such as wetting agents, emulsifying and suspending agents, sweetening, flavoring, coloring, perfuming and preservative agents. Suspensions, in addition to the one or more compounds according to the disclosure, may contain suspending agents as, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions disclosed herein, for rectal or vaginal administration may be presented as a suppository, which may be prepared by mixing the one or more compounds according to the disclosure, with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the disclosure. Pharmaceutical compositions which are suitable for vaginal administration also may include pessaries, tampons, creams, gels, pastes, foams or spray formulations containing carriers that are known in the art to be appropriate.

Dosage forms for the topical or transdermal administration of a pharmaceutical composition or pharmaceutical tablet of the present disclosure may include powders, sprays, ointments, pastes, creams, lotions, gels, solutions, patches and inhalants. The pharmaceutical composition or pharmaceutical tablet may be mixed under sterile conditions with a pharmaceutically acceptable carrier, and with any preservatives, buffers, or propellants which may be required.

The ointments, pastes, creams and gels may contain, in addition to the pharmaceutical composition or pharmaceutical tablet of the present disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a pharmaceutical composition or a pharmaceutical tablet of the present disclosure, excipients such as lactose, talc, silicic acid, aluminum hydroxide, calcium silicates and polyamide powder, or mixtures of these substances. Additionally, sprays may contain customary propellants, such as chlorofluorohydrocarbons and volatile unsubstituted hydrocarbons, such as butane and propane.

Ophthalmic formulations, eye ointments, powders, solutions and the like, are also contemplated as being within the scope of the present disclosure.

Compositions suitable for parenteral administration may comprise at least one more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In various embodiments, a composition described herein includes at least one compound chosen from compounds of formula (I) and pharmaceutically acceptable salts and solvates thereof and one or more surfactants. In some embodiments, the surfactant is sodium lauryl sulfate (SLS), sodium dodecyl sulfate (SDS), or one or more polyoxylglycerides. For example, the polyoxyglyceride can be lauroyl polyoxylglycerides (sometimes referred to as Gelucire™) or linoleoyl polyoxylglycerides (sometimes referred to as Labrafil™). Examples of such compositions are shown in PCT Patent Application No. PCT/US2014/033566, the contents of which are incorporated herein in its entirety.

As noted above, the methods disclosed herein may treat at least one disorder related to aberrant Stat3 pathway activity in a subject. Aberrant Stat3 pathway activity can be identified by expression of phosphorylated Stat3 (“pStat3”) or its surrogate upstream or downstream regulators.

The Stat3 pathway can be activated in response to cytokines, for example, IL-6, or by one or more tyrosine kinases, for example, EGFR, JAKs, Abl, KDR, c-Met, Src, and Her2. The downstream effectors of Stat3 include but are not limited to Bcl-xl, c-Myc, cyclinD1, Vegf, MMP-2, and survivin. The Stat3 pathway has been found to be aberrantly active in a wide variety of cancers, as shown in Table 1. Persistently active Stat3 pathway may occur in more than half of breast and lung cancers, hepatocellular carcinomas, multiple myelomas and in more than 95% of head and neck cancers. Blocking the Stat3 pathway causes cancer cell-growth arrest, apoptosis, and reduction of metastasis frequency in vitro and/or in vivo.

TABLE 1
DISEASES

| ONCOL- | Solid | Breast Cancer (Watson, C. J. and W. R. Miller. | |
|---|---|---|---|
| OGY | tumors | Br. J. Cancer, 1995. 71(4): p. 840-44) | |
| DIS- | | Head and Neck Cancer (SCCHN) (Song, J. I. | |
| EASES | | and J. R. Grandis. Oncogene, 2000. 19(21): p. | |
| | | 2489-95) | |
| | | Lung Cancer (Song, L., et al. Oncogene, 2003. | |
| | | 22(27): p. 4150-65) | |
| | | Ovarian Cancer (Savarese, T. M., et al. | |
| | | Cytokine, 2002. 17(6): p. 324-34) | |
| | | Pancreatic Cancer (Toyonaga, T., et al. | |
| | | Cancer Lett., 2003. 201(1): p. 107-16) | |
| | | Colorectal carcinoma (Corvinus, F. M., et al. | |
| | | Neoplasia, 2005. 7(6): p. 545-55) | |
| | | Prostate Cancer (Gao, B., et al. FEBS Lett., | |
| | | 2001.488(3): p. 179-84) | |
| | | Renal Cell carcinoma (Buettner, R., et al. Clin. | |
| | | Cancer Res., 2002. 8(4): p. 945-54) | |
| | | Melanoma (Carson, W. E. Clin. Cancer Res., | |
| | | 1998. 4(9): p. 2219-28) | |
| | | Hepatocellular carcinomas (Darnell, J. E. Nat. | |
| | | Med., 2005. 11(6): p. 595-96) | |
| | | Cervical Cancer (Chen, C. L., et al. Br. J. | |
| | | Cancer, 2007. 96(4): p. 591-99) | |
| | | Endometrial Cancer (Chen, C. L., et al. Br. J. | |
| | | Cancer, 2007. 96(4): p. 591-99) | |
| | | Sarcomas (Lai, R., et al. J. Pathol., 2006. | |
| | | 208(5): p. 624-32; and ) | |
| | | Brain Tumors (Punjabi, A. S., et al. J. Virol., | |
| | | 2007. 81(5): p. 2449-58) | |
| | | Gastric Cancers (Kanda, N., et al. Oncogene, | |
| | | 2004. 23(28): p. 4921-29) | |
| | Hemato- | Multiple Myeloma (Puthier, D., et al. Eur. J. | |
| | logic | Immunol., 1999. 29(12): p. 3945-50) | |
| | Tumors | Leukemia | HTLV-1-dependent Leukemia |
| | | | (Migone, T. S., et al. Science, |
| | | | 1995. 269(5220): p. 79-81) |
| | | | Chronic Myelogenous |
| | | | Leukemia (Buettner, R., et al. |
| | | | Clin. Cancer Res., 2002. 8(4): |
| | | | p. 945-54) |
| | | | Acute Myelogenous Leukemia |
| | | | (Spiekermann, K., et al. Eur. J. |
| | | | Haematol., 2001.67(2): p. 63- |
| | | | 71) |
| | | | Large Granular Lymphocyte |
| | | | Leukemia (Epling-Burnette, P. |
| | | | K., et al. J. Clin. Invest., 2001. |
| | | | 107(3): p. 351-62) |
| | | Lymphomas | EBV-related/Burkitt's (Weber- |
| | | | Nordt, R. M., et al. Blood, |
| | | | 1996. 88(3): p. 809-16) |
| | | | Mycosis Fungoides (Buettner, |
| | | | R., et al. Clin. Cancer Res., |
| | | | 2002. 8(4): p. 945-54) |
| | | | HSV Saimiri-dependent (T- |
| | | | cell) (Buettner, R., et al. Clin. |
| | | | Cancer Res., 2002. 8(4): p. |
| | | | 945-54) |
| | | | Cutaneous T-cell Lymphoma |
| | | | (Sommer, V. H., et al. |
| | | | Leukemia, 2004. 18(7): p. |
| | | | 1288-95) |
| | | | Hodgkin's Diseases (Buettner, |
| | | | R., et al. Clin. Cancer Res., |
| | | | 2002. 8(4): p. 945-54) |
| | | | Anaplastic Large-cell |
| | | | Lymphoma (Lai, R., et al. Am. |
| | | | J. Pathol., 2004. 164(6): p. |
| | | | 2251-58) |

In some embodiments, the at least one disorder may be chosen from cancers related to aberrant Stat3 pathway activity, such as colorectal cancer. Recent studies have disclosed cancer stem cells able to regenerate tumors. These cancer stem cells are disclosed to be functionally linked with continued malignant growth, cancer metastasis, recurrence, and cancer drug resistance. CSCs and their differentiated progeny appear to have markedly different biologic characteristics. They persist in tumors as a distinct, but rare population. Conventional cancer drug screenings depend on measurement of the amount of tumor mass and, therefore, may not identify drugs that act specifically on the CSCs. In fact, cancer stem cells have been disclosed to be resistant to standard chemotherapies and are enriched after standard chemotherapy treatments, which can result in refractory cancer and recurrence. Cancer stem cells have also been demonstrated to be resistant to radiotherapy. Baumann, M., et al. Nat. Rev. Cancer, 2008. 8(7): p. 545-54. The reported cancer types in which CSCs have been isolated include breast cancer, head cancer, neck cancer, lung cancer, ovarian cancer, pancreatic cancer, colorectal carcinoma, prostate cancer, melanoma, multiple myeloma, Kaposi sarcoma, Ewing's sarcoma, liver cancer, medulloblastoma, brain tumors, and leukemia. Stat3 has been identified as a CSCs survival and self-renewal factor. Therefore, Stat3 inhibitors may kill CSCs and/or may inhibit CSC self-renewal. According to some embodiments, cancer stem cell or cancer stem cells refer to a minute population of cancer stem cells that have self-renewal capability and are tumorigenic.

Disclosed herein are methods of inhibiting, reducing, and/or diminishing CSC survival and/or self-renewal comprising administering a therapeutically effective amount of at least one pharmaceutical composition comprising at least one compound of formula (I) in combination with a therapeutically effective regimen of FOLFIRI. Also disclosed herein are methods of inhibiting, reducing, and/or diminishing CSC survival and/or self-renewal comprising administering a therapeutically effective amount of at least one compound of formula (I) in combination with a therapeutically effective regimen of FOLFIRI.

Also disclosed herein are methods of treating at least one cancer that is refractory to a conventional chemotherapy and/or a targeted therapy in a subject comprising administering a therapeutically effective amount of at least one compound of formula (I) in combination with a therapeutically effective regimen of FOLFIRI. In some embodiments, the at least one compound is included in a pharmaceutical composition.

Disclosed herein are methods of treating recurrent cancer in a subject that has failed surgery, oncology therapy (e.g., chemotherapy), and/or radiation therapy, comprising administering a therapeutically effective amount of at least one compound of formula (I) in combination with a therapeutically effective regimen of FOLFIRI. In various embodiments, the at least one compound is included in a pharmaceutical composition.

Also disclosed herein are methods of treating or preventing cancer metastasis in a subject, comprising administering a therapeutically effective amount of at least one compound of formula (I) in combination with a therapeutically effective regimen of FOLFIRI. In various embodiments, the at least one compound is included in a pharmaceutical composition.

Disclosed herein are methods of treating cancer in a subject comprising administering a therapeutically effective amount of at least one compound of formula (I) in combination with a therapeutically effective regimen of FOLFIRI. In various embodiments, the at least one compound is included in a pharmaceutical composition.

In some embodiments, the cancer may be chosen from gastric and gastroesophageal adenocarcinoma, colorectal adenocarcinoma, breast cancer, ovarian cancer, head and neck cancer, and melanoma. In some embodiments, the cancer is advanced colorectal cancer (CRC). In some embodiments, the cancer is gastric adenocarcinoma. In some embodiments, the cancer is gastroesophageal adenocarcinoma.

In some embodiments, the cancer may be advanced. In some embodiments, the cancer may be refractory. In some embodiments, the cancer may be recurrent. In some embodiments, the cancer may be metastatic. In some embodiments, the cancer may be associated with overexpression of Stat3. In some embodiments, the cancer may be associated with nuclear β-catenin localization.

### EXAMPLES

The methods disclosed herein comprise administering to a subject in need thereof a therapeutically effective amount of at least one compound of formula (I) and a therapeutically effective regimen of FOLFIRI.

### Example 3

The effects of 2-acetylnaphtho[2,3-b]furan-4,9-dione in combination with FOLFIRI in patients with advanced gastrointestinal cancer, including CRC and gastric cancer, were studied in phase Ib open-label, multi-center studies. In one phase Ib open label, multi-center clinical study, the safety, tolerability, and preliminary anti-tumor activity of 2-acetylnaphtho[2,3-b]furan-4,9-dione in combination with FOLFIRI, with and without bevacizumab, were assessed in adult patients with advanced gastrointestinal cancer, including CRC and gastric cancer. In addition, the pharmacokinetic profile and the pharmacodynamics (biomarkers) of 2-acetylnaphtho[2,3-b]furan-4,9-dione in combination with FOLFIRI, with and without bevacizumab, were studied, and the recommended phase II dose (RP2D) of 2-acetylnaphtho[2,3-b]furan-4,9-dione and FOLFIRI, with or without bevacizumab, were evaluated.

In total, as of April 2015, 18 patients aged 40-72 were enrolled in the open label, multi-center, phase Ib study (see Table 2). These patients were pretreated with an average of >3 prior lines of therapy. Of this group, 10 patients (56%) previously progressed on FOLFIRI.

TABLE 2
Baseline Demographics & Laboratory Values
N = 18

| Number of Prior | N | % | Hemoglobin | | |
|---|---|---|---|---|---|
| 1-3 prior | 12 | 67% | Median | 12.6 | g/dL |
| >4 prior | 6 | 33% | Range | 10.3-16.5 | g/dL |
| Age | | | Neutrophils | | |
| Median | 56 | yrs | Median | 3.5 | 10⁹/L |
| Range | 40-72 | yrs | Range | 1.9-10.7 | 10⁹/L |
| Gender | N | % | Platelets | | |
| Female | 8 | 44% | Median | 195 | 10⁹u/L |
| Male | 10 | 56% | Range | 105-538 | 10⁹/L |
| Race | N | % | ALT | | |
| Caucasian | 16 | 89% | Median | 32 | U/L |
| Black | 1 | 6% | Range | 11-107 | U/L |
| Asian | 1 | 6% | | | |
| Kamofsky | N | % | AST | | |
| 100% | 2 | 11% | Median | 32 | U/L |
| 90% | 12 | 67% | Range | 16-88 | U/L |
| 80% | 3 | 17% | | | |
| 70% | 1 | 6% | | | |
| K-Ras Status | N | % | Creatinine | | |
| WT | 10 | 56% | Median | 0.8 | mg/dL |
| Mutant | 4 | 22% | Range | 0.4-1.1 | mg/dL |
| N/A | 4 | 22% | | | |

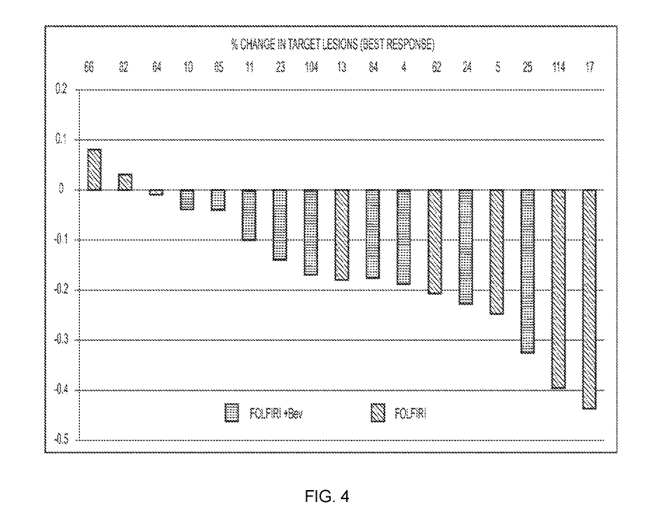

Of the 17 patients evaluable for response, 8 patients received 2-acetylnaphtho[2,3-b]furan-4,9-dione in combination with FOLFIRI and 9 patients received 2-acetylnaphtho[2,3-b]furan-4,9-dione in combination with FOLFIRI and bevacizumab (see FIG. 4).

Patients received continuous oral administration of 2-acetylnaphtho[2,3-b]furan-4,9-dione twice daily in 28 day cycles. A FOLFIRI regimen was administered (with or without bevacizumab depending on patient) every 14 days. Specifically, 2-acetylnaphtho[2,3-b]furan-4,9-dione was administered at a dose of 240 mg BID in combination with FOLFIRI (5-FU 400 mg/m² bolus with 2400 mg/m², irinotecan 180 mg/m², and leucovorin 400 mg/m² infusion) with or without bevacizumab 5 mg/kg, administered bi-weekly until progression of disease, unacceptable toxicity, or other discontinuation criterion was met.

Pharmacokinetics and pharmacodynamics were evaluated and objective tumor response was assessed every 8 weeks using Response Evaluation Criteria In Solid Tumors (RECIST 1.1).

This study demonstrated that 2-acetylnaphtho[2,3-b]furan-4,9-dione dosed at 240 mg BID was safely combined with FOLFIRI, with and without bevacizumab. Anti-cancer activity was observed in 94% of patients with advanced CRC who had failed prior standard chemotherapy. For example, as shown in Table 3, 94% (16 of 17) of evaluable patients had partial response (PR) or stable disease (SD). The Median progression-free survival (PFS) was 5.72 months. Additionally, 59% (10 of 17) of evaluable patients had prolonged SD (>6 months). And as demonstrated in FIG. 4, 88% (15 of 17) of evaluable patients had reduced target lesions.

Surprisingly, even for patients previously exposed to the FOLFIRI treatment but experiencing progression, the combination of 2-acetylnaphtho[2,3-b]furan-4,9-dione with bi-weekly FOLFIRI with or without bevacizumab reduced lesion formations in almost all of the patients. Without being limited to any particular theory, the presence of 2-acetylnaphtho[2,3-b]furan-4,9-dione appeared to re-sensitize the patients to the FOLFIRI treatment even when these patients had developed or started to develop resistance to the FOLFIRI treatment.

TABLE 3
Patient Summary

| | | # of Prior | | | | | |
|---|---|---|---|---|---|---|---|
| Patient | Chemotherapy | Lines of | Prior Line of Therapy Exposure | | | | |
| ID | Backbone | Therapy | FOLFIRI | Bevacizumab | Cetuximab | Panitumumab | Regorafenib |
| 25 | FOLFIRI | 4 | Exposed | Exposed | Naïve | Exposed | Exposed |
| 4 | FOLFIRI | 6 | Exposed | Exposed | Exposed | Naïve | Exposed |
| 17 | FOLFIRI | 3 | Exposed | Naïve | Exposed | Naïve | Naïve |
| 24 | FOLFIRI | 3 | Exposed | Exposed | Naïve | Naïve | Exposed |
| 66 | FOLFIRI | 2 | Exposed | Exposed | Exposed | Naïve | Naïve |
| 82 | FOLFIRI | 2 | Naïve | Naive | Naïve | Naïve | Naïve |
| 91 | FOLFIRI | 1 | Naïve | Exposed | Naïve | Naïve | Naïve |
| 104 | FOLFIRI | 1 | Naïve | Naïve | Naïve | Naïve | Naïve |
| 114 | FOLFIRI | 4 | Exposed | Naïve | Exposed | Naïve | Naïve |
| 5 | FOLFIRI/Bev | 1 | Naïve | Naïve | Naïve | Naïve | Naïve |
| 23 | FOLFIRI/Bev | 2 | Naïve | Naïve | Naïve | Naïve | Naïve |
| 13 | FOLFIRI/Bev | 11 | Exposed | Exposed | Naïve | Naïve | Exposed |
| 10 | FOLFIRI/Bev | 1 | Naïve | Exposed | Naïve | Naïve | Naïve |
| 11 | FOLFIRI/Bev | 6 | Exposed | Exposed | Exposed | Naïve | Exposed |
| 62 | FOLFIRI/Bev | 9 | Exposed | Exposed | Naïve | Naïve | Exposed |
| 64 | FOLFIRI/Bev | 3 | Exposed | Exposed | Naïve | Exposed | Naïve |
| 65 | FOLFIRI/Bev | 3 | Exposed | Exposed | Naïve | Naïve | Naïve |
| 84 | FOLFIRI/Bev | 1 | Naïve | Naïve | Naïve | Naïve | Naïve |

| Patient | Weeks | Best Response | |
|---|---|---|---|
| ID | on Study | (RECIST 1.1) | Comment |
| 25 | 36 | PR | 83% tumor regression, prolonged stable disease |
| 4 | 25 | SD | 19% tumor regression, prolonged stable disease |
| 17 | 17 | PR | 44% tumor regression |
| 24 | 16 | SD | 23% tumor regression |
| 66 | 6 | PD | 8% tumor growth, unequivocal progression of |
| | | | non-target lesions |
| 82 | 17 | SD | 3% tumor growth |
| 91 | 3 | N/A | N/A |
| 104 | 34+ | SD | 17 tumor regression, prolonged stable disease, |
| | | | continuing on study |
| 114 | 32+ | PR | 40% tumor regression, prolonged SD/PR, continuing |
| | | | on study |
| 5 | 85+ | SD | 25% tumor regression, prolonged stable disease, |
| | | | contuing on study |
| 23 | 29 | SD | 14% tumor regression, prolonged stable disease |
| 13 | 24 | SD | 18% tumor regression, prolonged stable disease |
| 10 | 18 | SD | 4% tumor regression |
| 11 | 15 | SD | 10% tumor regression |
| 62 | 14 | SD | 21% tumor regression |
| 64 | 25 | SD | 1% tumor regression; prolonged stable disease |
| 65 | 42 | SD | 4% tumor regression; prolonged stable disease |
| 84 | 25 | SD | 18% tumor regression; prolonged stable disease |

Gastrointestinal adverse events seen with 2-acetylnaphtho[2,3-b]furan-4,9-dione in combination with FOLFIRI with and without bevacizumab may be easily manageable with symptom medications.

As shown in Table 4, the most common adverse events included grade 1 and 2 diarrhea, abdominal pain, nausea, vomiting and anorexia. No dose limiting toxicity or new adverse events were seen, and the safety profile was similar to that of each regimen as monotherapy. Grade 3 events related to protocol therapy included diarrhea occurring in 3 patients, fatigue in 2 patients and dehydration in 1 patient. All events resolved after dose reduction and/or start of anti-diarrheal medications. No significant pharmacokinetic interactions were observed.

TABLE 4
Adverse Events (possibly/probably/definitely related)*
Number and Percent of Total Subjects with a given
Adverse Event by Grade
N = 18

| | | Grade 1 | | Grade 2 | | Grade 3 | |
|---|---|---|---|---|---|---|---|
| Organ System | Event | # | % | # | % | # | % |
| Gastrointestinal | Diarrhea | 18 | 100.0% | 9 | 50.0% | 3 | 16.7% |
| | Nausea | 14 | 77.8% | 6 | 33.3% | 0 | 0.0% |
| | Vomiting | 11 | 61.1% | 3 | 16.7% | 0 | 0.0% |
| | Abdominal | 7 | 38.9% | 4 | 22.2% | 0 | 0.0% |
| | Pain | | | | | | |
| | Flatulence | 2 | 11.1% | 1 | 5.6% | 0 | 0.0% |
| | Dry Mouth | 2 | 11.1% | 1 | 5.6% | 0 | 0.0% |
| Constitutional | Fatigue | 12 | 66.7% | 10 | 55.6% | 3 | 16.7% |
| | Weight Loss | 2 | 11.1% | 0 | 0.0% | 0 | 0.0% |
| | Platelet | 2 | 11.1% | 0 | 0.0% | 0 | 0.0% |
| | Count | | | | | | |
| Metabolism | Anorexia | 12 | 66.7% | 3 | 16.7% | 0 | 0.0% |
| Neuro-Psych | Dysgeusia | 5 | 27.8% | 0 | 0.0% | 0 | 0.0% |
| Renal & | Urine Dis- | 4 | 22.2% | 0 | 0.0% | 0 | 0.0% |
| Urinary | colorization | | | | | | |
| Dermatologic | Alopeda | 4 | 22.2% | 0 | 0.0% | 0 | 0.0% |
| Hematologic | Neutropenia | 3 | 16.7% | 2 | 11.1% | 0 | 0.0% |
| | Anemia | 2 | 11.1% | 2 | 11.1% | 0 | 0.0% |

In sum, the disclosed combination therapy provided for disease control (PR+SD) in 16 of 17 evaluable patients (94%) with 2 PR (per RECIST 1.1 criteria: 44% and 33% regression) and 14 SD (of which 13 (93%) had tumor regression <25%). In the evaluable patients, median progression free survival was 5.72 months. Of 17 patients, 7 (41%) had prolonged SD of >6 month.

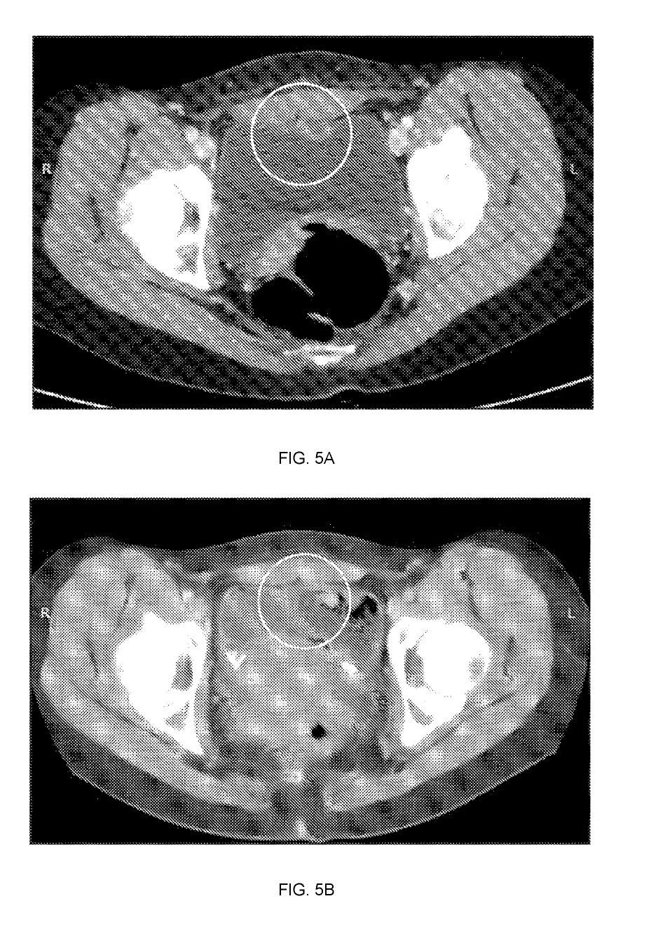

Among the subjects in the clinical trial, one was observed with complete response. This patient suffered from gastric adenocarcinoma with a metastatic lesion observed in the liver (FIG. 5A) and failed prior treatments of epirubicin, oxaliplatin, and Xeloda. After receiving the combination of 2-acetylnaphtho[2,3-b]furan-4,9-dione and FOLFIRI for 8 weeks, as shown in FIG. 5B, the metastatic lesion in liver was resolved.

In addition, patients were examined to determine whether cancer stem cell biomarkers are predictive of treatment outcome. Patients who were positive for the cancer stem cell marker pStat3 consistently exhibited longer median survival when treated with 2-acetylnaphtho[2,3-b]furan-4,9-dione in combination with FOLFIRI and bevacizumab compared to patients who were negative for pStat3. Without being limited to any particular theory, it would appear that pStat3 can serve as a predictive biomarker for prolonged survival.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described accordingly, all suitable modifications and equivalents may be resorted to, falling within the scope of the present disclosure. Other embodiments are within the following claims.

1-52. (canceled)

53. A method for treating colorectal cancer in a human subject comprising administering to said subject:
a compound having formula (I) 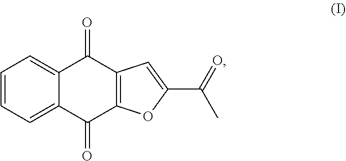 [SMILES: CC(=O)c1cc2c(o1)C(=O)c1ccccc1C2=O]
wherein the compound is administered orally to the subject at a total daily dose of about 480 mg; and
a therapeutically effective amount of: a 5-fluorouracil compound, a irinotecan compound, and a leucovorin compound (FOLFIRI).

54. The method according to claim 53, wherein the subject is administered a dose of about 240 mg of the compound of formula (I) twice daily.

55. The method according to claim 53, wherein the leucovorin compound is administered every 14 days as an about 400 mg/m² infusion.

56. The method according to claim 53, wherein the irinotecan compound is administered every 14 days as an about 180 mg/m² infusion.

57. The method according to claim 53, wherein the 5-fluorouracil compound is administered every 14 days as:
an about 400 mg/m² bolus infusion, followed by
an about 2400 mg/m² infusion.

58. The method according to claim 53, further comprising administering a therapeutically effective amount of bevacizumab.

59. The method according to claim 58, wherein bevacizumab is administered at a dose of about 5 mg/kg every 14 days.

60. The method according to claim 53, wherein the colorectal cancer is metastatic colorectal cancer.
